(19) United States
(12) Patent Application Publication
(10) Pub. No.: US 2026/0325450 A1
(43) Pub. Date: Oct. 22, 2026

(54) DRUG CARRIER FOR MUCOSAL ADMINISTRATION COMPRISING LYOPHILIZED HYALURONIC ACID-CARRYING DRUG

(71) Applicant: DONGGUK UNIVERSITY INDUSTRY-ACADEMIC COOPERATION FOUNDATION, Seoul (KR)

(72) Inventors: Choul Yong PARK, Goyang-si, Gyeonggi-do (KR); Mi Young JUNG, Seoul (KR)

(73) Assignee: DONGGUK UNIVERSITY INDUSTRY-ACADEMIC COOPERATION FOUNDATION, Seoul (KR)

(21) Appl. No.: 19/113,797

(22) PCT Filed: Sep. 19, 2023
(86) PCT No.: PCT/KR2023/014184

§ 371 Date: Mar. 20, 2025

(30) Foreign Application Priority Data
Sep. 21, 2022 (KR) 10-2022-0119081

(51) Int. Cl. A61K 9/20; A61K 31/5575

(52) U.S. Cl. CPC A61K 9/205; A61K 31/5575

(57) ABSTRACT

The present invention relates to a drug carrier for mucosal administration utilizing a drug-carrying lyophilized hyaluronic acid, a method for preparing the drug carrier, and a kit comprising the drug carrier. It is possible to deliver a drug to a precise location within a mucous membrane.

### CROSS-REFERENCE TO RELATED APPLICATIONS

This application is a 35 U.S.C. § 371 National Stage of International Patent Application No. PCT/KR2023/014184, filed Sep. 19, 2023, claiming benefit from Korean Patent Application No. 10-2022-0119081, filed Sep. 21, 2022, the disclosures of which are incorporated herein in their entirety by reference, and priority is claimed to each of the foregoing.

### TECHNICAL FIELD

The present invention relates to a drug delivery system for mucosal administration containing lyophilized hyaluronic acid carrying a drug.

The national research and development project that supported this invention is as follows
[National Research and Development Project That supported This Invention]
[Task Identification Number] 1465040345
[Subtask Number] HI23C0689000023
[Task Management Agency] Korea Health Industry Development Institute
[Research Project Title] Development of a Mucoadhesive Hyaluronic Acid Nanocarrier for Effective Delivery of Glaucoma Therapeutics
[Contribution Ratio] 1/1

### BACKGROUND

Hyaluronic acid is a linear polysaccharide composed of glucuronic acid and acetylglucosamine and is classified as one of the glycosamino glycans present in the extracellular matrix (ECM), synovial fluid and cartilage-supporting structures. It plays a crucial role as a signaling molecule in cell motility, differentiation, wound healing and cancer metastasis. In addition, hyaluronic acid is a biomaterial with excellent biocompatibility that can be used in tissue engineering and drug delivery systems because it has no immunologic problems. Hyaluronic acid typically has a molecular weight of between 1,000 and 10,000,000 Da, and has unique physicochemical properties and biological functions.

On the other hand, latanoprost, a lipid-soluble drug used as ophthalmic preparations to treat glaucoma, can be used at a concentration of 0.005%, which contains 5 μg per a drop, and it exhibits significantly low drug delivery efficiency. In addition, the current liquid eye drops have a high risk of side effects due to poor adherence and incorrect application in the elderly patients. In particular, prostaglandin-based eye drops for glaucoma treatment have a significant side effects of pigmentation in areas in contact with the skin.

The Korean Patent Application Laid-Open No. 10-2012-0118559 discloses a drug delivery composition comprising a biocompatible hyaluronic acid cross-linked product which is used in combination with other polymers, because hyaluronic acid alone is easily breakable and has limited usability.

A hyaluronic acid is used in the form of hydrogels to control the amount of release, but there has been no disclosure of a drug delivery system that administers a drug in the form of solid to the mucosa as in the present invention, rather than in the form of a liquid pharmaceutical composition.

Therefore, the present inventors completed the present invention by loading the lyophilized hyaluronic acid with a drug dissolved in ethanol, and drying it to obtain lyophilized hyaluronic acid loaded with a drug.

### SUMMARY OF THE INVENTION

An objective of the present invention is to provide a drug delivery system for mucosal administration containing lyophilized hyaluronic acid carrying a drug.

Another objective of the present invention is to provide a method for preparing the drug delivery system for mucosal administration comprising the lyophilized hyaluronic acid loaded with the drug.

An objective of the present invention is to provide a kit comprising the drug delivery system.

However, the technical problems to be achieved by the present invention are not limited to the above-mentioned problems, and other technical problems not mentioned may be clearly understood by those skilled in the art from the following description.

The terms used herein is for the purpose of description only and should not be construed as limiting. The singular forms include the plural forms unless the context clearly dictates otherwise. It should be understood that the terms “include” or “have” used herein specify the presence of stated features, numbers, steps, operations, elements, components, or combinations thereof, but do not preclude the presence or addition of one or more other features, numbers, steps, operations, elements, components, or combinations thereof. Unless otherwise defined, all terms used herein, including technical or scientific terms, have the same meaning as commonly understood by one of ordinary skill in the art to which the embodiment belongs. Terms such as those defined in commonly used dictionaries should be interpreted as having a meaning that is consistent with the meaning in the context of the relevant art and should not be interpreted in an idealized or overly formal sense unless expressly so defined herein.

Therefore, the present inventors have made extensive efforts to provide a technology necessary for a hyaluronic acid-based drug delivery system to effectively deliver a drug to various mucosal regions of the body, including eyeballs and conjunctiva. In specific embodiments, it was confirmed that for ophthalmic diseases, the drug could be efficiently delivered by simply contacting the conjunctiva, instead of eye drops that cause unnecessary administration. In other words, we have developed a new drug delivery system by contacting the mucous membrane with a solid from of drug rather than a liquid form of drug. When the solidified drug delivery system of the present invention is brought into contact with the mucosa, it is absorbed to the mucosa within seconds, enabling accurate drug delivery. This approach facilitates medication use for the elderly or motor-impaired patients, thereby improving treatment adherence and therapeutic efficacy.

To accomplish the above objectives, we developed a technology to carry lipid-soluble or water-soluble drugs, individually or in combination, using laboratory-scale lyophilized hyaluronic acid, and therefore evaluated their ocular treatment and intraocular pressure-lowering efficacy and stability in vitro and in vivo. The present invention is developed that the hyaluronic acid formulations is soluble in water, but not in solvents such as ethanol.

Accordingly, the present invention provides a drug delivery system for mucosal administration containing lyophilized hyaluronic acid carrying a drug.

The lyophilized hyaluronic acid may be prepared by a conventional lyophilization method and may be used in either a commercially available form or a manufactured form.

The drug is carried by the lyophilized hyaluronic acid, and this drug delivery system may be in a solidified form. Unlike conventional hyaluronic acid formulations by which are in liquid or gel form, the solidified hyaluronic acid allows for absorption upon contact. The form of the drug carried by the lyophilized hyaluronic acid includes all types in which a liquid drug is absorbed into the solid hyaluronic acid and subsequently dried, and may include homogeneously carried, layered, or included in a core portion, among other possible forms, without limitation.

The drug delivery system containing lyophilized hyaluronic acid loaded with a drug is in a solidified form obtained through lyophilization or cross-linking methods, and its internal structure may take various forms such as fibrous form, a mesh form, a lattice form, and a sponge form, but not limited thereto.

The drug delivery system may be prepared by (a) dissolving a drug, (b) loading the dissolved drug onto the lyophilized hyaluronic acid, and (c) drying the lyophilized hyaluronic acid loaded with the drug.

The drug delivery system may preferably include hyaluronic acid but not limited thereto and may include other polymer compounds that can serve as a solid drug delivery system. The polymer compound refers to a biocompatible material that aids in absorption or retention of the drug.

Additionally, the drug delivery system containing the lyophilized hyaluronic acid may further include one or more additives selected from commonly used carriers, excipients, disintegrants, sweeteners, coating agents, swelling agents, glidants, flavoring agents, antioxidants, buffer solutions, bacteriostatic agents, diluents, dispersing agents, surfactants, binders, and lubricants.

Specifically, carriers, excipients and diluents may include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinylpyrrolidone, water, methylparaben, propylparaben, talc, magnesium stearate, and mineral oil. In addition to simple excipients, lubricants such as magnesium stearate and talc may also be used. Formulations for mucosal administration may include sterile aqueous solutions, non-aqueous solvents, suspensions, emulsions, lyophilized preparations and suppositories.

The drug delivery system can be cut into various sizes depending on the target body part and may be used in combination with a handle if necessary. Currently, liquid-based ophthalmic drugs pose challenges such as poor compliance among the elderly patients and an increased risk of side effects due to improper administration, but since the present invention provides a solidified drug delivery system, it improves compliance among the elderly patients and minimizes skin complications caused by unintended contact with the skin when using liquid formulations.

Unlike conventionally used hydrogel-form hyaluronic acid, when the lyophilized hyaluronic acid of the present invention is used, it can come into contact with the mucosa and, upon contact, rapidly hydrates within seconds to deliver the carried drug.

In the drug delivery system for mucosal administration of the present invention, mucosal administration refers to a dosage form that is absorbed through the cavity mucosa. The mucosa may include conjunctival mucosa, corneal mucosa, tracheal mucosa, bronchial mucosa, gastrointestinal mucosa, vaginal mucosa, alveolar mucosa, and middle ear mucosa, and specifically, the mucosal administration may include intranasal inhalation through the nasal mucosa, ophthalmic administration onto the conjunctiva or cornea, intrapulmonary inhalation through the alveolar mucosa, inhalation targeting the tracheal or bronchial mucosa, intravaginal administration through the vaginal mucosa, oral or rectal administration through the gastrointestinal mucosa, including the stomach, duodenum, small intestine, colon, and rectum, and middle ear administration targeting a middle ear mucosa. Among these, the mucosa may be preferably a conjunctival or corneal mucosa, and the mucosal administration may preferably include ophthalmic administration onto the conjunctiva or cornea.

The formulation of the drug delivery system is not particularly limited as long as it can dissolve or disintegrate on the mucosa of various body cavities, and may be shaped according to the target administration site. In addition to tablets and dry powders, it may be in the form of a liquid formulation (including suspensions and amorphous gels) that can be filled into containers such as tubes or capsules.

The subjects for mucosal administration of the drug delivery system according to the present invention include humans and non-human mammals, and preferably humans. Examples of non-human mammals include, but are not limited to, mice, rats, hamsters, guinea pigs, rabbits, pigs, cattle, goats, sheep, dogs, cats, Himalayan monkeys, Philippine monkeys, orangutans, and chimpanzees.

It will be understood that the preferred dosage of the drug delivery system will be determined by the attending physician within the scope of reasonable medical judgment. The specific therapeutically effective dosage level for any particular subject will depend on various factors, including the disease, disorder, or condition being treated and its severity; the activity of the specific active ingredient used; the specific composition employed; the subject's age, weight, overall health, gender, and diet; the timing, route of administration, and excretion rate of the specific active ingredient; the duration of treatment; drugs used in combination with or simultaneously with the specific active ingredient; and factors well known in the medical field.

Accordingly, the drug delivery system can be absorbed within 1 to 15 seconds upon mucosa administration, and preferably within 1 to 10 seconds.

The drug carried by the drug delivery system of the present invention may include, but not limited to, glaucoma drugs, prostaglandin agents, anti-allergy drugs, steroid drugs, antibiotics, anti-inflammatory agents, and non-steroidal anti-inflammatory drugs.

In one aspect, the glaucoma drug may be one or more selected from timolol, brimonidine, acetazolamide, and betaxolol, but not limited thereto.

In one aspect, the prostaglandin agent may be one or more selected from latanoprost, bimatoprost, travoprost, and tafluprost, but not limited thereto.

In one aspect, the anti-allergy drug may be olopatadine, but not limited thereto.

In one aspect, the steroid drug may be one or more selected from fluorometholone and prednisolone, but not limited thereto.

Furthermore, the drug delivery system of the present invention may carry one or more drugs, enabling the effect of multiple drug delivery by a single drug delivery system.

The present invention may also provide a method for preparing the drug delivery system.

The preparation method may include:
(a) dissolving a drug;
(b) loading the dissolved drug onto lyophilized hyaluronic acid; and
(c) drying the lyophilized hyaluronic acid loaded with the drug.

Specifically, the drug delivery system comprising hyaluronic acid can carry a water-soluble drug by stirring it in the form of a liquid and solidifying it, while a lipid-soluble drug can be carried by incorporating the drug into the solidified drug delivery system through an additional process using a solvent capable of dissolving lipid-soluble drugs, such as oil or alcohol.

In one embodiment, the drug in the preparation method may be dissolved in ethanol.

In the method for preparing the drug delivery system for mucosal administration, the mucosa may be one or more selected from conjunctival mucosa, corneal mucosa, tracheal mucosa, bronchial mucosa, gastrointestinal mucosa, vaginal mucosa, alveolar mucosa, and middle ear mucosa, but not limited thereto.

In the method for preparing the drug delivery system for mucosal administration, the drug carried by the prepared drug delivery system may include, but not limited to, glaucoma drug, prostaglandin agent, anti-allergic drug, steroid drug, antibiotic, anti-inflammatory drug, and non-steroidal anti-inflammatory drug.

The description of the drug-related content is omitted to avoid redundancy with the aforementioned details.

In one embodiment, during step (c), drying may be carried out at a temperature of 15 to 40 ° C and a relative humidity of 48 to 55% for 15 to 120 minutes, and preferably, drying may be performed at a temperature of 20 to 35 ° C and a relative humidity of 48 to 55% for 30 to 90 minutes. If the process is conducted outside these ranges, proper drying may not occur, potentially resulting in an improperly solidified drug delivery system.

The present invention also provides a kit comprising the drug delivery system.

The kit may include filling materials for easy storage, desiccants considering the hygroscopicity of the drug, and cushioning materials for maintaining the shape, and formulations, packaging, and covers that facilitate the distribution of conventional pharmaceuticals may be used without limitation.

The drug delivery system of the present invention enables accurate mucosal localization of the drug and more effective localized delivery of the drug to the lesion than when using a liquid agent. In addition, it is easier to deliver the drug, which can improve compliance and increase therapeutic effects.

### BRIEF DESCRIPTIONS OF DRAWINGS

FIG. 1 shows the HPLC-DAD results evaluating whether the drug was successfully loaded in accordance with the intended amount.

FIG. 2 shows the intraocular pressure measurements for each experimental group: control group (●) Group 1 treated with saline (▪), Group 2 administered with eye drops (▾), Group 3 treated with lyophilized hyaluronic acid alone (▴), and Group 4 treated with the drug-loaded lyophilized hyaluronic acid of the present invention (♦)

FIGS. 3A-3B show the histological analysis performed to assess the presence of inflammation or tissue injury following drug administration in each group.

FIG. 4 shows the RT-PCR results obtained after RNA extraction from the samples, confirming the expression of inflammation-related genes.

FIGS. 5A-5D show a preparation example involving the drug delivery system of the present invention.

### DETAILED DESCRIPTION

The following examples are provided to facilitate a better understanding of the present invention and will be described in detail. However, these examples are merely illustrative and do not limit the scope of the present invention. Rather, they are presented to enable those skilled in the art to more fully comprehend the invention.

### Preparation Example

### Formulation of Lyophilized Hyaluronic Acid Drug Delivery System

Accordingly, the inventors loaded a drug, pre-dissolved in EtOH, onto lyophilized hyaluronic acid, followed by a subsequent drying process to obtain drug-loaded lyophilized hyaluronic acid.

Latanoprost (Sigma-Aldrich) was dissolved in 100% ethanol to prepare a 0.1% (1 mg/mL) latanoprost solution, which was subsequently loaded onto 1 mg of lyophilized hyaluronic acid (Genicos, Korea) in the desired amount. Following drug loading, the samples were dried under ambient conditions in a clean bench at a relative humidity of approximately 48-55% for 1 hour.

### Example 1

### HPLC-DAD (High Performance Liquid Chromatography—Diode Array Detection) Analysis

After incorporating the target drug amount into the drug delivery system, HPLC analysis was performed to evaluate whether the loaded drug was successfully retained within the lyophilized hyaluronic acid at the intended concentration.

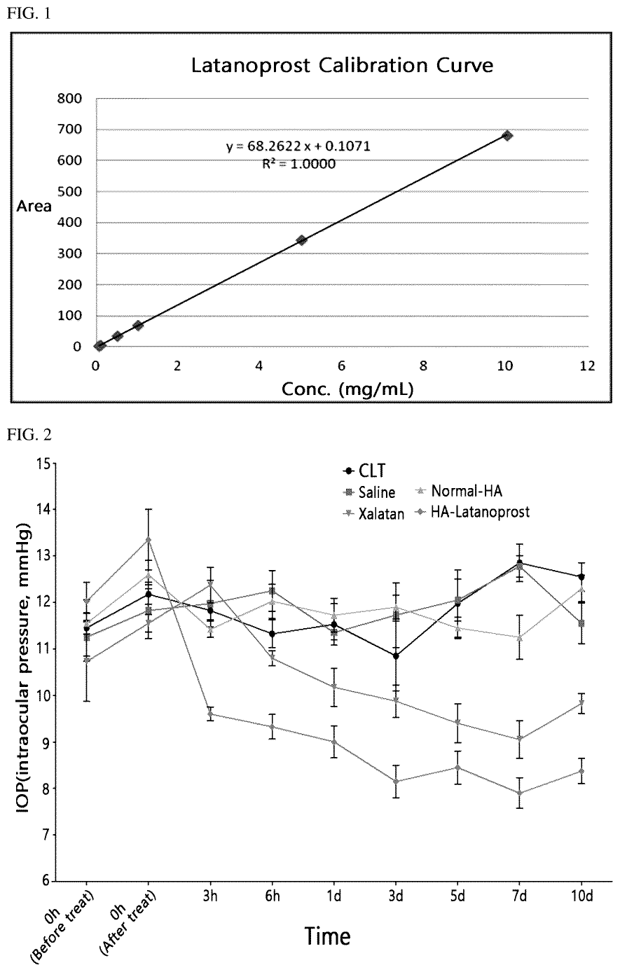

FIG. 1 presents the HPLC-DAD (High Performance Liquid Chromatography—Diode Array Detection) results obtained from the Korea Polymer Research Institute. Latanoprost, used as a standard substance, was dissolved in absolute EtOH at a concentration of 1 mg/mL=0.1%. As a result, a standard calibration curve was obtained with an R² value of 1.0000.

### Example 2

### Optimization of Samples

The composition and sample names of the samples are shown in [Table 1] below.

TABLE 1

| | | sample name used |
|---|---|---|
| No. | sample name | in the test |
| 1 | HA Tablet (aqueous) Latanoprost 34.9 ug | Koptri-22-07-01195-1 |
| 2 | HA tablet (water-soluble) containing | Koptri-22-07-01195-2 |
| | 34.2 μg of latanoprost. | |

The sample existed in a solid form as a bulk mass and in a liquid form as an aqueous solution. 1. Lyophilized tablet form: HA (34.9 mg)+latanoprost (34.9 μg loaded and dried).

2. Lyophilized tablet form: HA (34.2 mg)+latanoprost (34.2 μg loaded and dried).

The test methods and results are as follows.

TABLE 2

| sample | Analysis | | Analytical | Detection | Analysis |
|---|---|---|---|---|---|
| name | Parameters | Unit | Method | Limit | Results |
| Koptri-22- | Latanoprost | mg/kg | HPLC- | 0.05 | 0.961 |
| 07-01195-1 | | | DAD | | |
| Koptri-22- | Latanoprost | mg/kg | HPLC- | 0.05 | 0.919 |
| 07-01195-2 | | | DAD | | |

The elution of the HPLC-DAD (High Performance Liquid Chromatography-Diode Array Detection) sample was performed using a shaker at 30 ° C, 250 rpm for 3 hours in 4 mL of ethanol, followed by analysis. During sample analysis, the dilution factor within the analytical instrument was 9.9502.

Accordingly, the amount of latanoprost in each sample is as follows.

TABLE 3

| | | Amount of |
|---|---|---|
| | (Analysis result) * (Dilution factor for elution | latanoprost |
| No. | conditions) * (Dilution factor during analysis) | in the sample |
| 1 | 0.961 * 4(Dilution factor for elution conditions) * | 38.25 ug |
| | 9.9502(Dilution factor during analysis) | |
| 2 | 0.919 * 4(Dilution factor for elution conditions) * | 36.58 ug |
| | 9.9502(Dilution factor during analysis) | |

This experiment was conducted to verify whether the actual loaded drug amount could be detected at the same level through LC analysis. As a result, it was confirmed that most samples contained the intended drug amount, and confirmed that there was almost no loss of drug prepared by this method.

### Example 3

### Validation of Safety and Efficacy Through Animal Experiments

A total of 20 BL6 mice were classified into the following groups.

TABLE 4

| Group | n | R | L |
|---|---|---|---|
| Group 1 | 5 | CTL | Saline 5 uL |
| Group 2 | 5 | CTL | Eye drops (Zalatan) 5 uL ophthalmic solution |
| | | | (Zalatan Ophthalmic Solution 50 μg/mL, 0.005%), |
| | | | containing 0.25 μg latanoprost |
| Group 3 | 5 | CTL | Lyophilized HA 1 mg |
| Group 4 | 5 | CTL | Lyophilized HA 1 mg + 0.25 μg of latanoprost |
| | | | (prepared by applying 0.25 μL of 1 μg/μL |
| | | | latanoprost solution and drying) |

Each group received instillations and drops at the same time each day after the start of the experiment. IOP (Intraocular pressure) was measured at 0 h (baseline), immediately after treatment, 1 h, 3 h, 6 h, and on days 1, 3, 5, 7, 10, and 14.

The i-care IC100 (Revenio Group) was used. To minimize the influence of anesthesia, all measurements were conducted without anesthesia using a portable tono meter. Additionally, to reduce variations in intraocular pressure due to diurnal fluctuations, measurements were consistently conducted at the same time each day following drug administration.

As a result, FIG. 2 illustrates the changes in IOP. The IOP measurements for the control group (●), Group 1 treated with saline (▪), Group 2 administered with eye drops (▾), Group 3 treated with lyophilized hyaluronic acid alone (▴), and Group 4 treated with drug-loaded lyophilized hyaluronic acid of the present invention (♦) indicate that the TOP in Group 4 was significantly lower compared to the other groups. This indicates that Group 4, which was treated with the drug-loaded lyophilized hyaluronic acid of the present invention, consistently exhibited a superior IOP-lowering effect compared to the group administered with Xalatan eye drops.

Additionally, it was confirmed that the application in mice could be performed without anesthesia, causing no pain and no ocular injury.

### Example 4

### LC-MS Analysis Data

After drug administration in each group, tear samples were collected and analyzed using LC-MS to quantify the actual amount of drug delivered. Comparative Examples 1-1 and 1-2 refer to the samples collected from Group 2 (▾), which was treated with Xalatan eye drops, whereas Examples 1-1 and 1-2 refer to the samples collected from Group 4 (♦), which was treated with the drug-loaded lyophilized hyaluronic acid of the present invention.

TABLE 5

| | Analysis | | Analytical | Detection | Analysis |
|---|---|---|---|---|---|
| Sample | Parameters | Unit | Method | Limit | Results |
| Comparative | latanoprost | mg/kg | HPLC- | 0.05 | Not |
| Example1-1 | | | DAD | | detected |
| Comparative | latanoprost | mg/kg | HPLC- | 0.05 | Not |
| Example11-2 | | | DAD | | detected |
| Example 1-1 | latanoprost | mg/kg | HPLC- | 0.05 | 2.5 |
| | | | DAD | | |
| Example 1-2 | latanoprost | mg/kg | HPLC- | 0.05 | 2.9 |
| | | | DAD | | |

As a result, even after accounting for the dilution factor, it was confirmed that only the HA-latanoprost treatment group retained approximately 10~17.24% of the drug in tears one minute after administration. In contrast, no detectable drug was observed in the Xalatan eye drop treatment group.

### Example 5

### Histological Examination

After drug administration in each group, a histological examination was conducted to assess the presence of inflammation or damage. Histology was analyzed by H&E staining after tissue sections.

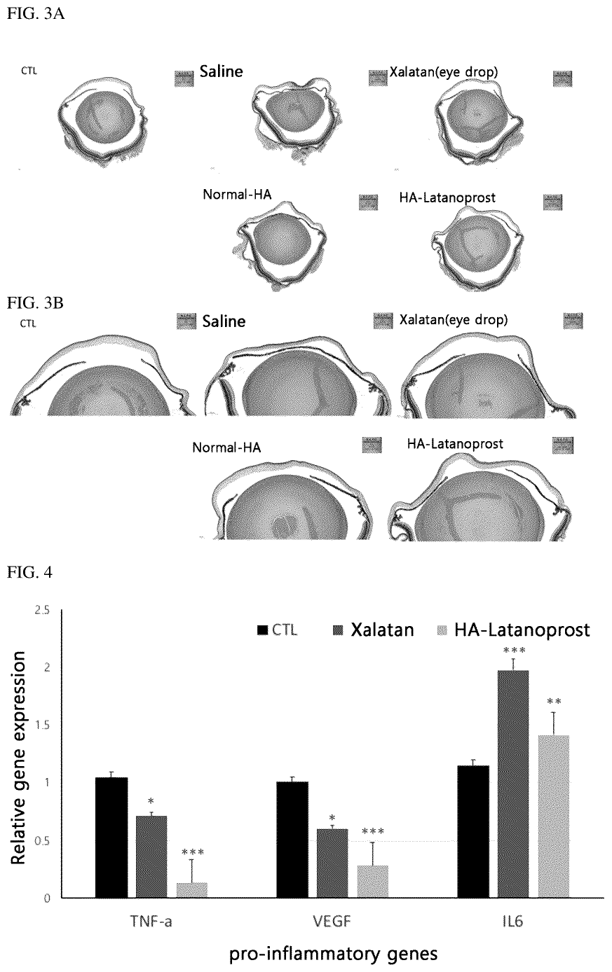

In FIGS. 3A-3B, CTL represents the control group (●) for all experimental groups, the control group for the Xalatan-treated group corresponds to Group 1 (▪), which was treated with saline, while the control group for the HA-latanoprost-treated group corresponds to Group 3 (▴), which was treated with lyophilized hyaluronic acid only (Normal HA).

As a result, as shown in FIG. 3A, no signs of inflammation, injury, or damage were observed in the histological examination of any group.

Additionally, as shown in FIG. 3B, even when magnified to focus on the peripheral corneal region where the drug was administered, no signs of inflammation, injury, or damage were observed in the histological examination of any group.

### Example 6

### RT-PCR Results

RNA was extracted from the collected tear samples, followed by RT-PCR analysis.

The expression levels of inflammatory-inducing genes, including TNF-α, VEGF, and IL-6, were analyzed. As shown in FIG. 4, the HA-latanoprost group of the present invention exhibited a significant reduction in the expression of inflammation-related genes compared to the control group and the group administered with Xalatan eye drops containing the same concentration of latanoprost.

### Manufacturing Example 1

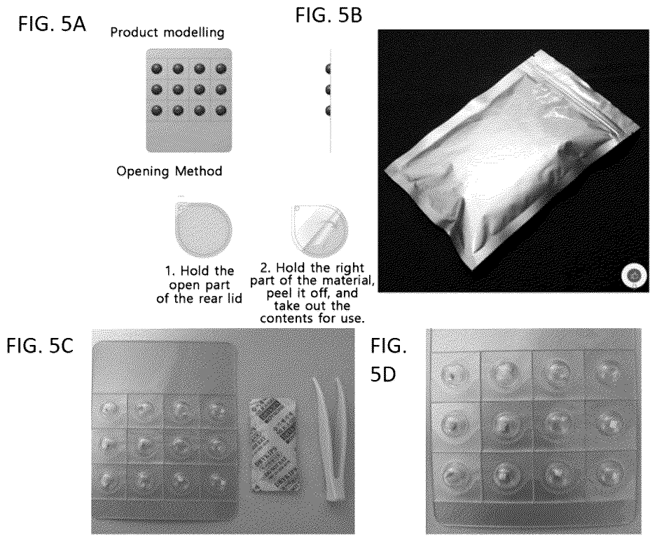

The drug delivery system containing the lyophilized hyaluronic acid of the present invention was packaged in a PTP (press-through package) using a 5 mm-diameter transparent capsule silver foil cover, considering the hygroscopicity of the drug (FIG. 5A). Subsequently, the product is further packaged in a silver foil zipper bag along with a silica gel desiccant (FIG. 5B). The rear lid can be peeled off by holding the open part, allowing the contents to be removed and used.

1. A drug delivery system for mucosal administration, containing lyophilized hyaluronic acid carrying a drug.

2. The drug delivery system of claim 1, wherein the drug delivery system is solidified.

3. The drug delivery system of claim 1, wherein the internal structure in the lyophilized hyaluronic acid has one or more selected from a group consisting of a fibrous form, a mesh form, a lattice form, and a sponge form.

4. The drug delivery system of claim 1, wherein the drug delivery system is prepared by:
(a) dissolving a drug;
(b) loading the dissolved drug onto lyophilized hyaluronic acid; and
(c) drying the lyophilized hyaluronic acid loaded with the drug.

5. The drug delivery system of claim 1, wherein the mucosa in the mucosal administration include one or more selected from a group consisting of conjunctiva, cornea, tracheal mucosa, bronchial mucosa, gastrointestinal mucosa, vaginal mucosa, alveolar mucosa, and middle ear mucosa.

6. The drug delivery system of claim 1, wherein the drug delivery system is absorbed within 1 to 15 seconds upon mucosa administration.

7. The drug delivery system of claim 1, wherein the drug includes one or more selected from a group consisting of an anti-glaucoma drug, a prostaglandin agent, an anti-allergic drug, a steroid drug, an antibiotic, an anti-inflammatory drug, and a non-steroidal anti-inflammatory drug.

8. The drug delivery system of claim 7, wherein the anti-glaucoma drug includes one or more selected from a group consisting of timolol, brimonidine, acetazolamide, and betaxolol.

9. The drug delivery system of claim 7, wherein the prostaglandin agent includes one or more selected from a group consisting of latanoprost, bimatoprost, travoprost, and tafluprost.

10. The drug delivery system of claim 7, wherein the anti-allergic drug includes olopatadine.

11. The drug delivery system of claim 7, wherein the steroid drug includes one or more selected from a group consisting of fluorometholone and prednisolone.

12. A method for preparing the drug delivery system according to claim 1.

13. The method of claim 12, wherein the method comprises:
(a) dissolving a drug;
(b) loading the dissolved drug onto lyophilized hyaluronic acid; and
(c) drying the lyophilized hyaluronic acid loaded with the drug.

14. The method of claim 13, wherein the drug is dissolved in ethanol at (a).

15. The method of claim 13, wherein the drying is performed at a temperature of 15 to 40 ° C, at a relative humidity of 48 to 55%, for 15 to 120 minutes at (c).

16. A kit comprising the drug delivery system according to claim 1.
